(19) United States
(12) Patent Application Publication
(10) Pub. No.: US 2025/0216402 A1
(43) Pub. Date: Jul. 3, 2025

(54) BIOMARKERS FOR SEVERITY OF ISCHEMIC STROKE AND USE THEREOF

(71) Applicant: Zhejiang Chinese Medical University, Zhejiang (CN)

(72) Inventors: Haitong Wan, Zhejiang (CN); Huifen Zhou, Zhejiang (CN); Yu He, Zhejiang (CN); Jiehong Yang, Zhejiang (CN)

(73) Assignee: Zhejiang Chinese Medical University, Zhejiang (CN)

(21) Appl. No.: 18/657,726

(22) Filed: May 7, 2024

(30) Foreign Application Priority Data
Dec. 29, 2023 (CN) 202311846546.4

(51) Int. Cl. G01N 33/68; G01N 33/92

(52) U.S. Cl. CPC G01N 33/6896; G01N 33/92; G01N 2333/4703; G01N 2333/70539; G01N 2333/90206; G01N 2333/924; G01N 2333/98; G01N 2333/99; G01N 2800/2871

(57) ABSTRACT

The present invention discloses biomarkers for the severity of ischemic stroke, belonging to the field of biomedicine. The first aspect of the invention provides biomarkers for the severity of ischemic stroke, including protein markers, small molecule metabolite markers, and lipid metabolite markers. The second aspect of the invention provides the use of these biomarkers. The innovation of the biomarkers lies in their ability to provide more accurate diagnostic results, thereby assisting clinicians in better treatment planning. Additionally, these biomarkers offer advantages such as ease of operation and low cost, making them potentially widely applicable in clinical settings.

### CROSS-REFERENCE TO RELATED APPLICATION

This application claims the priority benefit of China application serial no.202311846546.4, filed on Dec. 29, 2023. The entirety of the above-mentioned patent application is hereby incorporated by reference herein and made a part of this specification.

### BACKGROUND

### Technical Field

The present invention pertains to the field of biomedicine, specifically involving a biomarker for the severity of ischemic stroke and its application.

### Description of Related Art

Ischemic stroke, referred to as ischemic apoplexy in traditional Chinese medicine, poses a severe threat to human life and quality of survival. It is characterized by high mortality, incidence, disability, and recurrence rates, with mortality, incidence, and disability ranking among the top three. The high disability and recurrence rates impose a substantial burden on society. Ischemic stroke (IS) accounts for approximately 50% to 65% of cerebrovascular diseases, making it a significant focus of medical attention globally.

The pathogenesis of ischemic stroke is complex, and in Western medicine, treatment primarily revolves around antiplatelet and thrombolytic therapies. However, these treatments may lead to side effects such as bleeding, neurotoxicity, and reocclusion, and their efficacy remains to be improved. Traditional Chinese medicine approaches the treatment of ischemic stroke by targeting multiple pathological processes, intervening actively through various pathways, targets, and levels. This approach offers distinct advantages, including precise efficacy and minimal side effects.

However, there have been relatively few biomarkers available for the diagnosis of stroke over an extended period. Diagnostic methods for patients with mild brain injuries such as concussion, traumatic brain injury, and stroke primarily rely on techniques such as computed tomography (CT) and magnetic resonance imaging (MRI). CT scans are routinely employed to assist physicians in evaluating brain injuries, enabling rapid, straightforward, and accurate exclusion of cerebral hemorrhage, subdural hematoma, and tumor strokes. However, the use of CT scans is limited, especially for significant injuries with mild symptoms. In certain situations, such as the onset of cerebral infarction, lesions may not be evident in the brain during CT detection. Lesions smaller than 1 cm are challenging to detect with CT, and their location may be elusive. Damaged brain tissue often shares a density similar to normal brain tissue, making routine CT scans prone to false negatives, necessitating enhanced scanning. Statistical data from literature reports indicate a significant proportion of patients with mild brain injuries or concussions presenting at emergency departments yield negative CT scan results, with a positive diagnostic rate for cerebral infarction ranging from 60% to 84%.

In order to have clinical utility, new diagnostic biomarkers, when used as single markers, should ideally surpass or be at least equivalent to other known markers in the field. The value of a new marker is demonstrated in terms of its sensitivity and specificity. Finding a serum marker that balances both sensitivity and specificity and can be applied in clinical detection of stroke is urgently needed to achieve early diagnosis, postoperative monitoring, and prognosis assessment in stroke management.

### SUMMARY

In response to the challenges present in existing technologies, the purpose of the present invention is to provide a biomarker for the severity of ischemic stroke and its application. This is achieved through the following technical solutions:

Aspect 1 of the invention introduces a biomarker for the severity of ischemic stroke, encompassing protein markers. The protein markers include ACOX3, CAP1, GRIPAP1, MAN1A1, UQCRC1, SULT1A2, VNN1, BPGM, COPS8, HLA-DRB3, SPATS2L, VPS26A, and DOCK1.

Furthermore, it includes small molecule metabolite markers, with the small molecule metabolite markers comprising Valylleucine, (1S,2R,4As,6aS,6bR,8R,9R,10R,11R,12aR,12bR,14bS)-8,10,11-trihydroxy-9-(hydroxymethyl)-1,2,6a,6b,9,12a-hexamethyl-1,2,3,4,4a,5,6,6a,6b,7,8,8a,9,10,11,12,12a,12b,13,14b-icosahydropicene-4a-carboxylic acid, 1R-cis-3,3,5-Trimethylcyclohexyl ester 5-oxo-L-proline, Olopatadine n-oxide, Tetramethylene sulfoxide, Phenylacetaldehyde, Pentrinitrol, 13-OxoODE, 2-[[(2S)-1-[[(2S)-2-Carboxy-2-hydroxyethyl]amino]-1-oxo-3-phenylpropan-2-yl]amino]-4-phenylbutanoic acid, Beta-Citryl-L-glutamic acid, Desmethylmianserin, N,N-Didesmethyltramadol, Triethylene glycol dimethacrylate, and Uracil mustard.

Moreover, it further includes lipid metabolite markers, with the lipid metabolite markers comprising 2-eicosyl-3-hydroxy-34-carboxy-tetratriacontanoic acid, 3beta-hydroxy-4beta-methyl-5alpha-cholest-7-ene-4alpha-carboxylic acid, 6-pentadecyl salicylic acid, AC2SGL(16:0/28:0(2Me[S],4Me[S],6Me[S],8Me[R],10Me[R],11OH)), Am-PE(16:0/20:5(5Z,8Z,11Z,14Z,17Z)), Am-PE(18:0/22:6(4Z,7Z,10Z,13Z,16Z,19Z)), Artonin P, CL(1′-[16:0/18:0],3′-[16:0/16:0]), DG(21:0/22:6(4Z,7Z,10Z,13Z,16Z,19Z)/0:0)[iso2], ethyl 2E,4Z-decadienoate, LacCer(d18:1/18:1(9Z)), LacCer(d18:1/22:0), lanosteryloleate, N-(2-fluoro-ethyl) arachidonoyl amine, PC(O-20:0/19:1(9Z)), PI(O-20:0/0:0), PIM1(16:0/18:0), PS(14:1(9Z)/18:3(6Z,9Z,12Z)), and Sitostanyl-22:0.

The second aspect of the present invention involves the application of the aforementioned biomarkers in the preparation of diagnostic kits for assessing the severity of ischemic stroke.

This invention pertains to a biomarker for the severity of ischemic stroke, characterized by its ability to provide more accurate diagnostic results, assisting physicians in formulating effective treatment plans. Furthermore, this biomarker exhibits advantages such as simplicity of operation and cost-effectiveness, making it potentially widely adopted in clinical applications.

### BRIEF DESCRIPTION OF THE DRAWINGS

FIG. 1A, FIG. 1B, FIG. 1C, FIG. 1D, FIG. 1E and FIG. 1F depict the biomarker prediction for the severity of ischemic stroke (IS) in Example 1.

FIG. 1A. Flowchart of the random forest prediction model analysis for IS severity in different populations.

FIG. 1B. Distribution of populations with varying severity of IS in the training and validation groups.

FIG. 1C. Core proteins displayed based on the model's prediction results.

FIG. 1D. PCA analysis of core proteins showing distinct boundaries between populations with different severity levels of IS.

FIG. 1E. AUC results based on proteins.

FIG. 1F. Boxplots illustrating the expression patterns of core proteins in different severity groups of IS, analyzed through Kruskal-Wallis test.

FIG. 2A, FIG. 2B, FIG. 2C, FIG. 2D, FIG. 2E and FIG. 2F present the biomarker prediction for the severity of IS in Example 2.

FIG. 2A. Flowchart of the random forest prediction model analysis for IS severity in different populations.

FIG. 2B. Distribution of populations with varying severity of IS in the training and validation groups.

FIG. 2C. Core small molecule metabolites displayed based on the model's prediction results.

FIG. 2D. PCA analysis of core small molecule metabolites showing distinct boundaries between populations with different severity levels of IS.

FIG. 2E. AUC results based on small molecule metabolites.

FIG. 2F. Boxplots illustrating the expression patterns of core small molecule metabolites in different severity groups of IS, analyzed through Kruskal-Wallis test.

FIG. 3A, FIG. 3B, FIG. 3C, FIG. 3D, FIG. E and FIG. 3F showcase the biomarker prediction for the severity of IS in Example 3.

FIG. 3A. Flowchart of the random forest prediction model analysis for IS severity in different populations.

FIG. 3B. Distribution of populations with varying severity of IS in the training and validation groups.

FIG. 3C. Core lipid metabolites displayed based on the model's prediction results.

FIG. 3D. PCA analysis of core lipid metabolites showing distinct boundaries between populations with different severity levels of IS.

FIG. 3E. AUC results based on lipid metabolites.

FIG. 3F. Boxplots illustrating the expression patterns of core lipid metabolites in different severity groups of IS, analyzed through Kruskal-Wallis test.

### DESCRIPTION OF THE EMBODIMENTS

Further clarification of the present invention is provided below in conjunction with the accompanying description and figures to enhance a better understanding of the technical solution.

### Example 1: Screening of Protein Biomarkers for Ischemic Stroke Severity

### Method:

### 1. Participant Recruitment and Study Design

This study enrolled a total of 143 participants aged 40-90 years, including 123 patients with ischemic stroke collected from 9 centers between August 2018 and October 2020, and an additional 20 normal healthy controls. The study adhered to the Helsinki Declaration and relevant clinical trial research standards in China, registered at the China Clinical Trial Registry with ID ChiCTR1800015189. Each participant voluntarily joined the trial and signed an informed consent form. Patients were grouped based on different criteria:

Severity grouping according to the National Institutes of Health Stroke Scale (NIHSS) scores: ≤4 for mild, 5≤NIHSS score≤9 for mild to moderate, and 10≤NIHSS score≤15 for moderate.

### 2. Inclusion and Exclusion Criteria

### Inclusion Criteria:

(1) Individuals meeting the diagnostic criteria for ischemic stroke in Western medicine.
(2) Individuals diagnosed with the syndrome of meridians and collaterals obstruction, qi deficiency, and blood stasis in traditional Chinese medicine for stroke.
(3) Patients confirmed by imaging studies (CT or MRI) to have anterior circulation infarction (OCSP clinical classification as total anterior circulation infarct or partial anterior circulation infarct).
(4) Onset duration: 1 to 4 weeks after the occurrence of symptoms.
(5) Assessment using the National Institutes of Health Stroke Scale (NIHSS) with scores ranging from 4 to 20.
(6) First onset or prior history of cerebral infarction without pre-existing disability (Modified Rankin Scale score≤1).
(7) Age between 40 and 90 years (inclusive), with no gender restrictions.
(8) Voluntary participation and signed informed consent by the individual.

### Exclusion Criteria:

(1) Individuals with a bleeding tendency or those who experienced bleeding within the last 3 months, including post-stroke depression, dementia, or complications such as cerebral hemorrhage after cerebral infarction. Also, exclusion of patients with posterior circulation infarction (POCI), transient ischemic attack (TIA), intracranial abnormal vascular network disease, asymptomatic cerebral infarction, or those with unstable vital signs due to extensive cerebral infarction.
(2) Individuals who discontinued traditional Chinese medicine for ischemic stroke for less than 1 week or those who received thrombolytic therapy after the current episode.
(3) Participants with other conditions affecting limb function, including but not limited to limping, osteoarthritis, rheumatoid arthritis, gouty arthritis, which may interfere with neurological function examination.
(4) Those confirmed by examination to have hemiplegia caused by brain tumors, traumatic brain injury, cerebral cysticercosis, or embolic stroke caused by atrial fibrillation associated with valvular heart disease, coronary heart disease, or other heart diseases.
(5) Individuals unable to independently complete daily activities due to various diseases and physical weakness before the current illness, severely affecting the efficacy evaluation.
(6) Participants with other serious cardiovascular, cerebral, hepatic, renal, hematopoietic system diseases, recent major surgeries, or those unsuitable for participation in this study.
(7) Patients with psychiatric disorders, severe depression, alcohol dependence, or a history of drug abuse.
(8) Pregnant or lactating women, those planning to conceive within 3 months after medication or within 3 months after discontinuation of medication.
(9) Individuals with allergies or a history of allergic reactions to multiple drugs, or those allergic to components of the investigational drugs.
(10) Participants who participated in other clinical studies in the 3 months prior to the trial.
(11) Patients deemed unsuitable for participation in this study by the investigators.

### 3. Protein Extraction and Mass Spectrometric Analysis for Data-Independent Acquisition (DIA)

Two microliters of plasma sample were mixed with 98 μL of 50 mM ABC buffer and heat-inactivated at 95° C. for 3 minutes. Subsequently, under the condition of enzyme-to-protein ratio being 1:25, peptides were generated through trypsin digestion at 37° C., followed by collection and drying using a SpeedVac system (Eppendorf). The peptide samples were then loaded onto a 2 cm self-packed trapping column (inner diameter 100 μm, 3 μm ReproSil-Pur C18-AQ beads, Dr. MaiSch GmbH) with solvent A (0.1% formic acid in water) and separated on a chromatographic column (inner diameter 150 μm, length 15 cm, 1.9 μm ReproSil-Pur C18-AQ beads, Dr. MaiSch GmbH). The eluted peptides were ionized at 2 kV and introduced into the mass spectrometer.

Protein components in the samples were analyzed using a Q Exactive HF-X hybrid quadrupole-Orbitrap mass spectrometer (Thermo Fisher Scientific) coupled with a liquid chromatography system (EASY nLC 1200, Thermo Fisher Scientific). Data-independent acquisition (DIA) was employed for mass spectrometric analysis. DIA scans were performed with a resolution of 60 k (AGC target 4e5 or 50 ms) in the range of 300 to 1,400 m/z for MS1 scans. Subsequently, 30 DIA fragments were obtained at a resolution of 15 k, with an AGC target of 5e4 or a maximum injection time of 22 ms. The normalized collision energy for HCD fragmentation was set to 27%. Spectra were captured in profile mode. MS2 default charge state was set to 3. Qualitative and quantitative analysis of the raw MS data were performed using the iProteome one-stop data analysis cloud platform.

4. Data Processing: Missing values (NAs) in this study were handled using the match between runs (MBR) algorithm, widely applied in proteomics and considered an effective approach for imputing missing values. We established a dynamic regression function based on commonly identified peptide segments in the samples. The regression function was employed to calculate the retention time (RT) of the corresponding hidden peptides. Linear or quadratic functions were selected based on the R2 correlation value. The existence of the extracted ion chromatogram (XIC) was verified based on m/z and the calculated RT. The peak area values of XIC obtained through this function were considered part of the respective protein. This approach has been applied in other proteomic studies.

Machine Learning: The Random Forest (RF) classifier, a supervised learning algorithm based on ensemble trees, was employed to predict biomarkers related to the severity of stroke. The 123 IS patients were divided into a training set and a test set with a ratio of 7:3. Parameters were set as ntree=500, mtry=100, and the final RF model was generated through training. The importance function was invoked to analyze the average accuracy and average Gini coefficient of all independent variables. This provided insights into the contribution of each molecule to the predictive ability of the model, sorted from high to low importance, and identified the core molecules. The randomForest R package (version 3.7-1.1) was used for these analyses.

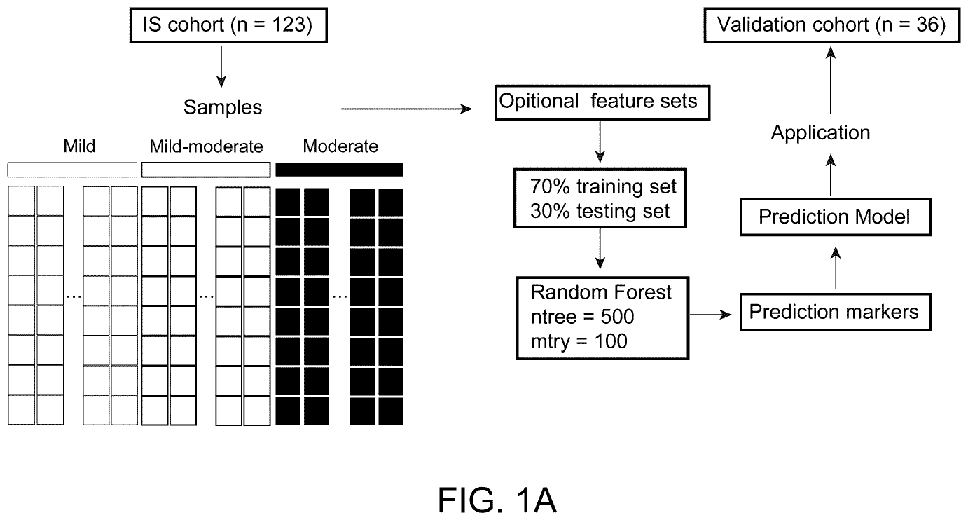

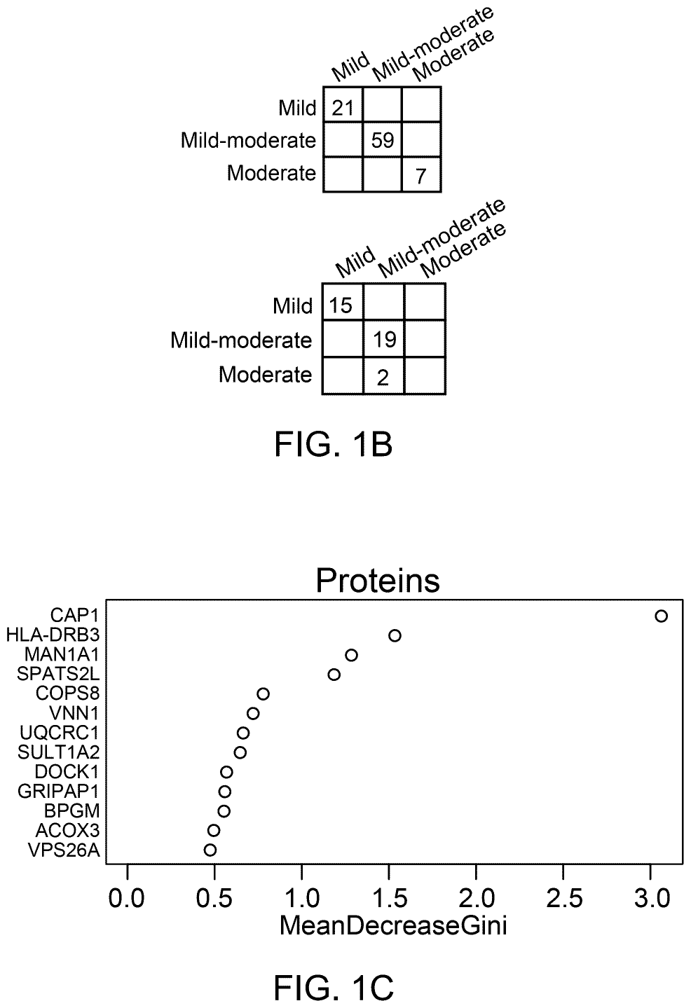

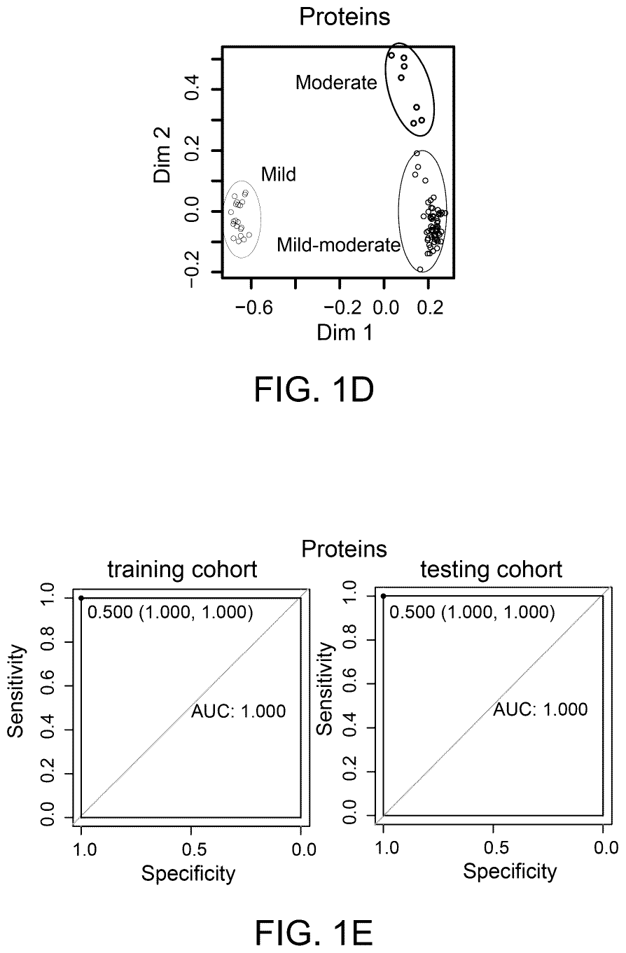

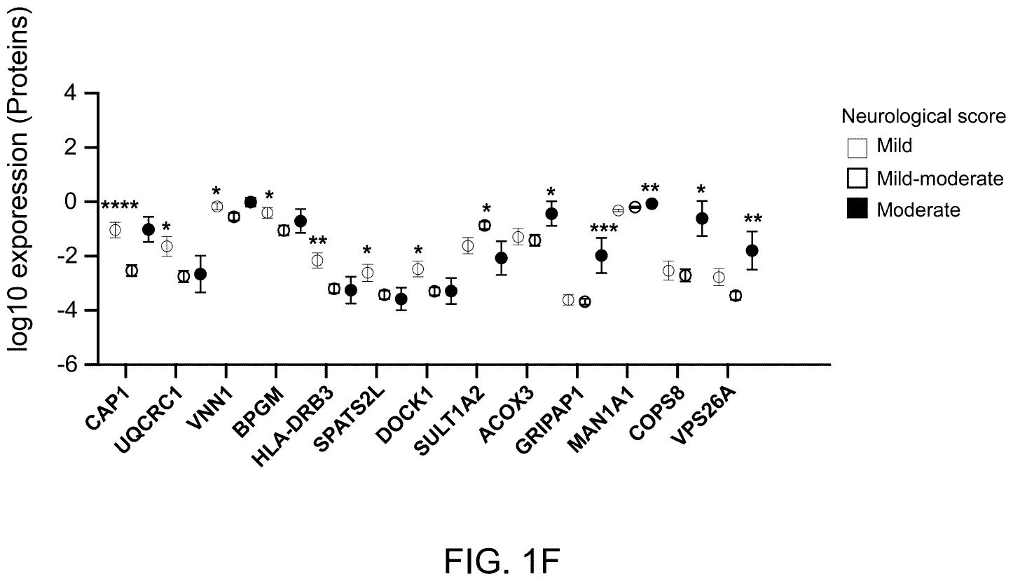

Results: As illustrated in FIG. 1A, FIG. 1B, FIG. 1C, FIG. 1D, FIG. 1E and FIG. 1F, we identified a set of core proteins serving as potential biomarkers associated with the severity of ischemic stroke (IS). These proteins include ACOX3, CAP1, GRIPAP1, MANIA1, UQCRC1, SULT1A2, VNN1, BPGM, COPS8, HLA-DRB3, SPATS2L, VPS26A, and DOCK1.

### Example 2: Screening of Small Molecule Metabolite Markers for Different Degrees of Disease in Ischemic Stroke

### Method:

### 1. Participant Recruitment And Study Design

### Participant Recruitment and Study Design

This study enrolled a total of 143 participants aged 40-90 years, including 123 patients with ischemic stroke collected from 9 centers between August 2018 and October 2020, and an additional 20 normal healthy controls. The study adhered to the Helsinki Declaration and relevant clinical trial research standards in China, registered at the China Clinical Trial Registry with ID ChiCTR1800015189. Each participant voluntarily joined the trial and signed an informed consent form. Patients were grouped based on different criteria:

Severity grouping according to the National Institutes of Health Stroke Scale (NIHSS) scores: ≤4 for mild, 5≤NIHSS score≤9 for mild to moderate, and 10≤NIHSS score≤15 for moderate.

### 2. Inclusion and Exclusion Criteria

### Inclusion Criteria:

(1) Individuals meeting the diagnostic criteria for ischemic stroke in Western medicine.
(2) Individuals diagnosed with the syndrome of meridians and collaterals obstruction, qi deficiency, and blood stasis in traditional Chinese medicine for stroke.
(3) Patients confirmed by imaging studies (CT or MRI) to have anterior circulation infarction (OCSP clinical classification as total anterior circulation infarct or partial anterior circulation infarct).
(4) Onset duration: 1 to 4 weeks after the occurrence of symptoms.
(5) Assessment using the National Institutes of Health Stroke Scale (NIHSS) with scores ranging from 4 to 20.
(6) First onset or prior history of cerebral infarction without pre-existing disability (Modified Rankin Scale score≤1).
(7) Age between 40 and 90 years (inclusive), with no gender restrictions.
(8) Voluntary participation and signed informed consent by the individual.

### Exclusion Criteria:

(1) Individuals with a bleeding tendency or those who experienced bleeding within the last 3 months, including post-stroke depression, dementia, or complications such as cerebral hemorrhage after cerebral infarction. Also, exclusion of patients with posterior circulation infarction (POCI), transient ischemic attack (TIA), intracranial abnormal vascular network disease, asymptomatic cerebral infarction, or those with unstable vital signs due to extensive cerebral infarction.
(2) Individuals who discontinued traditional Chinese medicine for ischemic stroke for less than 1 week or those who received thrombolytic therapy after the current episode.
(3) Participants with other conditions affecting limb function, including but not limited to limping, osteoarthritis, rheumatoid arthritis, gouty arthritis, which may interfere with neurological function examination.
(4) Those confirmed by examination to have hemiplegia caused by brain tumors, traumatic brain injury, cerebral cysticercosis, or embolic stroke caused by atrial fibrillation associated with valvular heart disease, coronary heart disease, or other heart diseases.
(5) Individuals unable to independently complete daily activities due to various diseases and physical weakness before the current illness, severely affecting the efficacy evaluation.
(6) Participants with other serious cardiovascular, cerebral, hepatic, renal, hematopoietic system diseases, recent major surgeries, or those unsuitable for participation in this study.
(7) Patients with psychiatric disorders, severe depression, alcohol dependence, or a history of drug abuse.
(8) Pregnant or lactating women, those planning to conceive within 3 months after medication or within 3 months after discontinuation of medication.
(9) Individuals with allergies or a history of allergic reactions to multiple drugs, or those allergic to components of the investigational drugs.
(10) Participants who participated in other clinical studies in the 3 months prior to the trial.
(11) Patients deemed unsuitable for participation in this study by the investigators.

### 3. Metabolite Extraction and Analysis of LC-MS/MS

After slow thawing at 4° C., 50 μL of plasma was pipetted into a 1.5 mL centrifuge tube. Subsequently, 300 μL of pre-cooled methanol/water solution (2:1) was added to the tube, and the tissue was homogenized by vortexing for 30 seconds. Following this, the tube was further vortexed for an additional 30 seconds with 600 μL of MTBE solution. The tube was then placed in a cold bath and exposed to 10 minutes of ultrasound to aid extraction. Subsequently, centrifugation was performed at 14,000 g and 4° C. for 10 minutes. The resulting solution had two layers, with the bottom layer containing the metabolite sample. This layer was separated and collected into different EP tubes. The samples were then vacuum-dried at room temperature and stored at −80° C.

Separation of the samples was achieved using the Nexera UHPLC LC-30A microflow ultra-high-performance liquid chromatography system. Initially, 98% of mobile phase A was used to equilibrate the chromatographic column. The samples were then transferred to an HILIC column (Waters, ACQUITY UPLC BEH Amide 1.7 μm, 2.1×100 mm column) at a flow rate of 0.3 mL/min using an autosampler. Over the next 11.5 minutes, the gradient elution was carried out, linearly increasing from 2% to 98% of mobile phase B. The gradient elution proceeded as follows: starting from 2% of mobile phase B, holding for 0.5 minutes, increasing to 98% of mobile phase B over 4 minutes, adjusting back to 2% of mobile phase B in 0.1 minutes, and then performing a 1.9-minute wash step.

Following chromatographic separation, sample evaluation was conducted using the Q Exactive HF-X mass spectrometer in both positive and negative ion modes. The full scan range for the parent ions was set at 70 to 1050 m/z. For MS1, the resolution was set at 120,000, automatic gain control (AGC) target at 3e6, and maximum injection time (max IT) at 100 ms. For MS2, with an AGC target of 2e5 and a max IT of 50 ms, the resolution was set at 7500. Fragmentation was performed using High-Energy Collision Dissociation (HCD) mode, with normalized collision energy set at 20, 40, and 60. The sub-ion scan range was 200 to 2000 m/z, and the isolation window was set at 1.5 m/z. Raw data collected from mass spectrometry analysis were processed using Progenesis QI software in raw file format. This software facilitated database retrieval for obtaining identification information of the analyzed samples.

4. Data Processing: In the present study, missing values (NAs) were addressed using a match between runs (MBR) algorithm, widely applied in proteomics and considered an effective method for filling missing values. We employed a dynamic regression function based on commonly identified peptide sequences in the samples. The regression function calculated the retention time (RT) of the corresponding hidden peptides, selecting linear or quadratic functions based on the magnitude of the correlation coefficient R\hat{ }2. An examination of m/z and calculated RT verified the existence of ion chromatograms (XIC) extracted through this process. The XIC peak area values obtained through this function were considered representative of the corresponding proteins. This approach has previously been utilized in other proteomic studies.

5. Machine Learning: The random forest (RF) classifier, a supervised learning algorithm based on ensemble trees, was employed to predict biomarkers related to the severity of stroke. A total of 123 IS patients were divided into training and test sets at a ratio of 7:3. The model was trained with parameters set as ntree=500, mtry=100, generating the final random forest model. The importance function was utilized to analyze the mean accuracy and mean Gini coefficient of all variables, ranking the molecules based on their contribution to the predictive ability of the model. The implementation of this process utilized the randomForest R package (version 3.7-1.1).

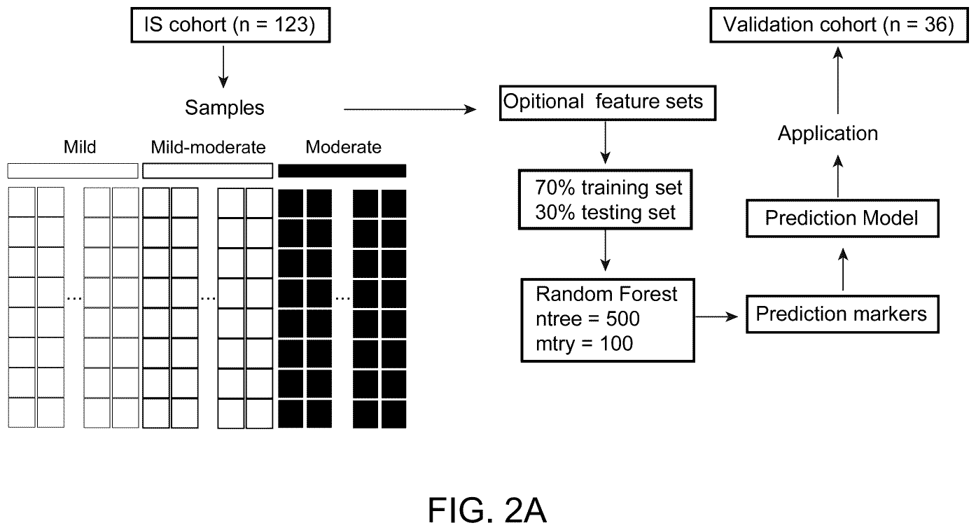

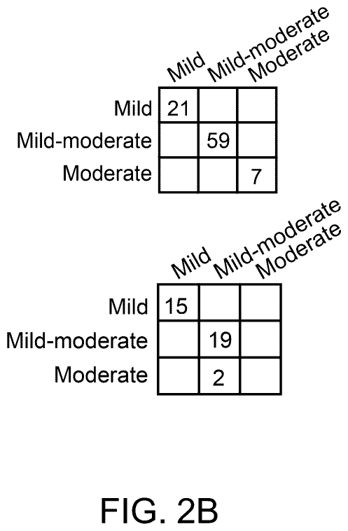

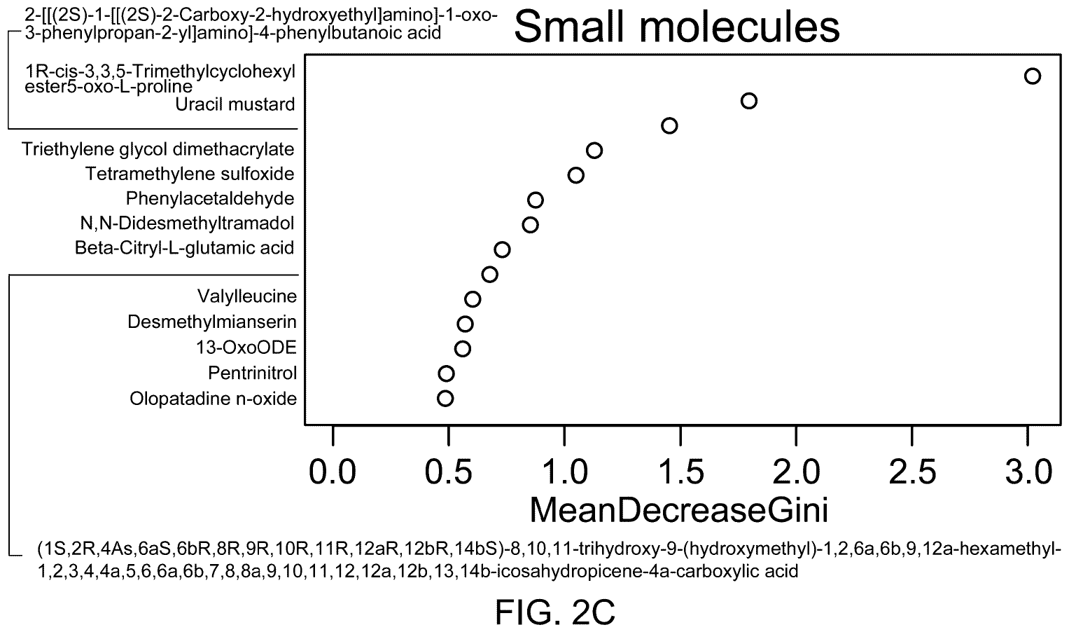

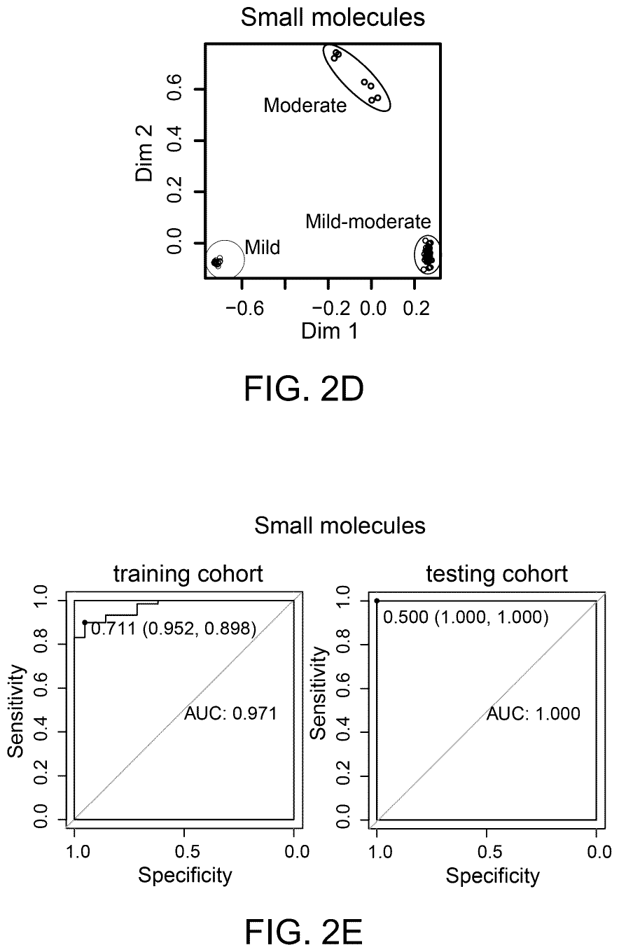

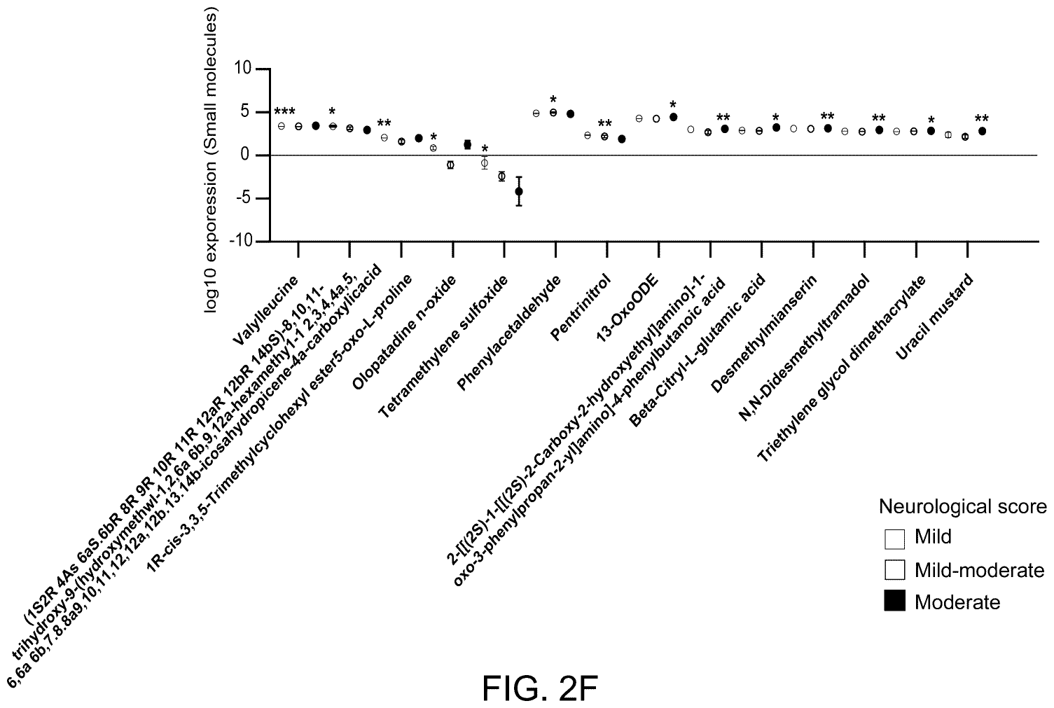

Results: As illustrated in FIG. 2A, FIG. 2B, FIG. 2C, FIG. 2D, FIG. 2E and FIG. 2F, a set of core small molecules was identified as potential biomarkers related to the severity of IS. These molecules include Valylleucine, (1S,2R,4As,6aS,6bR,8R,9R,10R,11R,12aR,12bR,14bS)-8,10,11-trihydroxy-9-(hydroxymethyl)-1,2,6a,6b,9,12a-hexamethyl-1,2,3,4,4a,5,6,6a,6b,7,8,8a,9,10,11,12,12a,12b,13,14b-icosahydropicene-4a-carboxylic acid, 1R-cis-3,3,5-Trimethylcyclohexyl ester5-oxo-L-proline, Olopatadine n-oxide, Tetramethylene sulfoxide, Phenylacetaldehyde, Pentrinitrol, 13-OxoODE, 2-[[(2S)-1-[[(2S)-2-Carboxy-2-hydroxyethyl]amino]-1-oxo-3-phenylpropan-2-yl]amino]-4-phenylbutanoic acid, Beta-Citryl-L-glutamic acid, Desmethylmianserin, N,N-Didesmethyltramadol, Triethylene glycol dimethacrylate, and Uracil mustard.

### Example 3: Screening of lipid metabolite markers for the severity of ischemic stroke

### Method:

### 1. Participant Recruitment and Study Design

This study enrolled a total of 143 participants aged 40-90 years, including 123 patients with ischemic stroke collected from 9 centers between August 2018 and October 2020, and an additional 20 normal healthy controls. The study adhered to the Helsinki Declaration and relevant clinical trial research standards in China, registered at the China Clinical Trial Registry with ID ChiCTR1800015189. Each participant voluntarily joined the trial and signed an informed consent form. Patients were grouped based on different criteria:

Severity grouping according to the National Institutes of Health Stroke Scale (NIHSS) scores: ≤4 for mild, 5≤NIHSS score≤9 for mild to moderate, and 10≤NIHSS score≤15 for moderate.

### 2. Inclusion and Exclusion Criteria

### Inclusion Criteria:

(1) Individuals meeting the diagnostic criteria for ischemic stroke in Western medicine.
(2) Individuals diagnosed with the syndrome of meridians and collaterals obstruction, qi deficiency, and blood stasis in traditional Chinese medicine for stroke.
(3) Patients confirmed by imaging studies (CT or MRI) to have anterior circulation infarction (OCSP clinical classification as total anterior circulation infarct or partial anterior circulation infarct).
(4) Onset duration: 1 to 4 weeks after the occurrence of symptoms.
(5) Assessment using the National Institutes of Health Stroke Scale (NIHSS) with scores ranging from 4 to 20.
(6) First onset or prior history of cerebral infarction without pre-existing disability (Modified Rankin Scale score≤1).
(7) Age between 40 and 90 years (inclusive), with no gender restrictions.
(8) Voluntary participation and signed informed consent by the individual.

### Exclusion Criteria:

(1) Individuals with a bleeding tendency or those who experienced bleeding within the last 3 months, including post-stroke depression, dementia, or complications such as cerebral hemorrhage after cerebral infarction. Also, exclusion of patients with posterior circulation infarction (POCI), transient ischemic attack (TIA), intracranial abnormal vascular network disease, asymptomatic cerebral infarction, or those with unstable vital signs due to extensive cerebral infarction.
(2) Individuals who discontinued traditional Chinese medicine for ischemic stroke for less than 1 week or those who received thrombolytic therapy after the current episode.
(3) Participants with other conditions affecting limb function, including but not limited to limping, osteoarthritis, rheumatoid arthritis, gouty arthritis, which may interfere with neurological function examination.
(4) Those confirmed by examination to have hemiplegia caused by brain tumors, traumatic brain injury, cerebral cysticercosis, or embolic stroke caused by atrial fibrillation associated with valvular heart disease, coronary heart disease, or other heart diseases.
(5) Individuals unable to independently complete daily activities due to various diseases and physical weakness before the current illness, severely affecting the efficacy evaluation.
(6) Participants with other serious cardiovascular, cerebral, hepatic, renal, hematopoietic system diseases, recent major surgeries, or those unsuitable for participation in this study.
(7) Patients with psychiatric disorders, severe depression, alcohol dependence, or a history of drug abuse.
(8) Pregnant or lactating women, those planning to conceive within 3 months after medication or within 3 months after discontinuation of medication.
(9) Individuals with allergies or a history of allergic reactions to multiple drugs, or those allergic to components of the investigational drugs.
(10) Participants who participated in other clinical studies in the 3 months prior to the trial.
(11) Patients deemed unsuitable for participation in this study by the investigators.

### 3. Metabolite Extraction and Analysis of LC-M S/MS

After slow thawing at 4° C., 50 μL of plasma was pipetted into a 1.5 mL centrifuge tube. Subsequently, 300 μL of pre-cooled methanol/water solution (2:1) was added to the tube, and the tissue was vortexed for 30 seconds for homogenization. Then, 600 μL of MTBE solution was used for an additional 30 seconds of vortexing. The tube was placed in a cold bath and exposed to 10 minutes of ultrasound to aid extraction. Afterward, centrifugation was performed at 14,000 g, 4° C. for 10 minutes. The resulting solution had two layers, with the upper layer containing lipids, which were separated and collected in different EP tubes. Subsequently, the samples were vacuum-dried at room temperature and stored at −80° C.

The separation of samples was carried out using the Nexera UHPLC LC-30A microflow ultra-high-performance liquid chromatography system. Initially, 98% of mobile phase A was used to equilibrate the chromatographic column. The samples were then transferred to a lipid column (Thermo, Acclaim C30, 3 μm, 2.1×100 mm column) at a flow rate of 0.26 mL/min using an automatic sampler. Over the next 5 minutes, the gradient elution increased linearly from 30% to 100% mobile phase B, following the gradient elution as follows: starting from 30% mobile phase B, maintain for 20 minutes. After 5 minutes at 100% mobile phase B, adjust back to 30% mobile phase B for 0.1 minutes, followed by a 2.9-minute wash step.

Following chromatographic separation, the samples were evaluated using the Q Exactive HF-X mass spectrometer in both positive and negative ion modes. The full scan range for parent ions was set at 200-2000 m/z. For MS1, the resolution was set at 120,000, with an automatic gain control (AGC) target of 1e6 and maximum injection time (max IT) set to 100 ms. For MS2, with an AGC target of 2e5 and max IT of 80 ms, the resolution was set at 15,000. High-energy collision dissociation (HCD) was employed for fragmentation, with normalized collision energies set at 20, 40, and 60. The isolation window was set at 1.5 m/z. Raw data collected from mass spectrometry analysis were processed using Progenesis QI software, facilitating database retrieval for identification information of the analyzed samples.

4. Data Processing: In addressing missing values (NAs) in this study, a match between runs (MBR) algorithm was employed, widely used in proteomics and considered an effective method for filling missing values. A dynamic regression function based on commonly identified peptide sequences in the samples was established. The regression function calculated the retention time (RT) of the corresponding hidden peptides, selecting linear or quadratic functions based on the magnitude of the correlation coefficient R\hat{ }2. An examination of m/z and calculated RT verified the existence of ion chromatograms (XIC) extracted through this process. The XIC peak area values obtained through this function were considered representative of the corresponding proteins. This approach has previously been utilized in other proteomic studies.

5. Machine Learning: The random forest (RF) classifier, a supervised learning algorithm based on ensemble trees, was employed to predict biomarkers related to the severity of stroke. A total of 123 IS patients were divided into training and test sets at a ratio of 7:3. The model was trained with parameters set as ntree=500, mtry=100, generating the final random forest model. The importance function was utilized to analyze the mean accuracy and mean Gini coefficient of all variables, ranking the molecules based on their contribution to the predictive ability of the model. The implementation of this process utilized the randomForest R package (version 3.7-1.1).

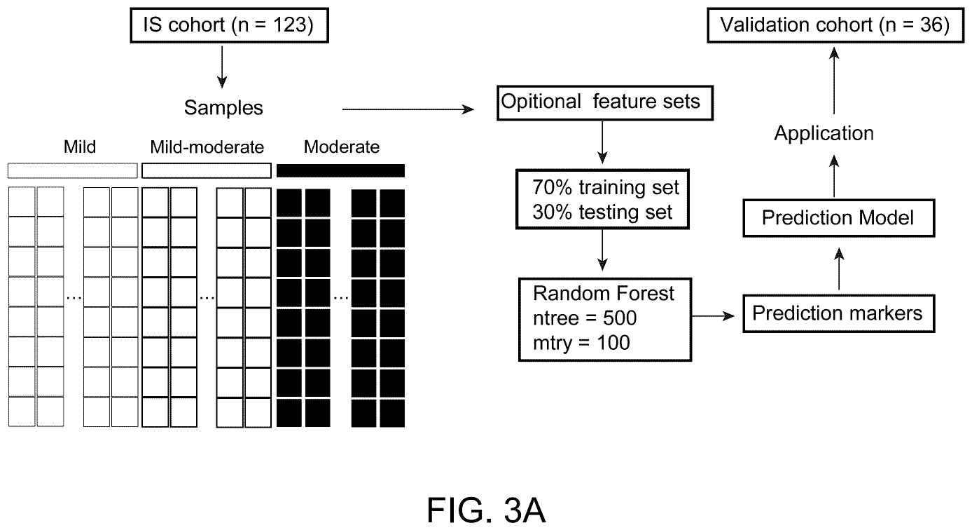

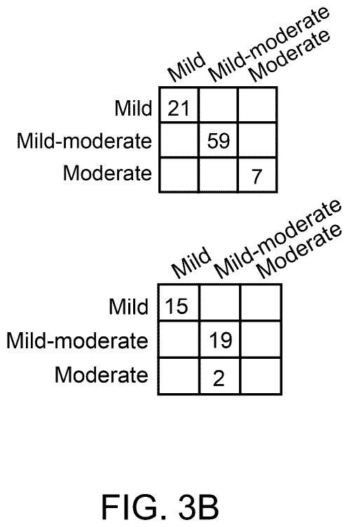

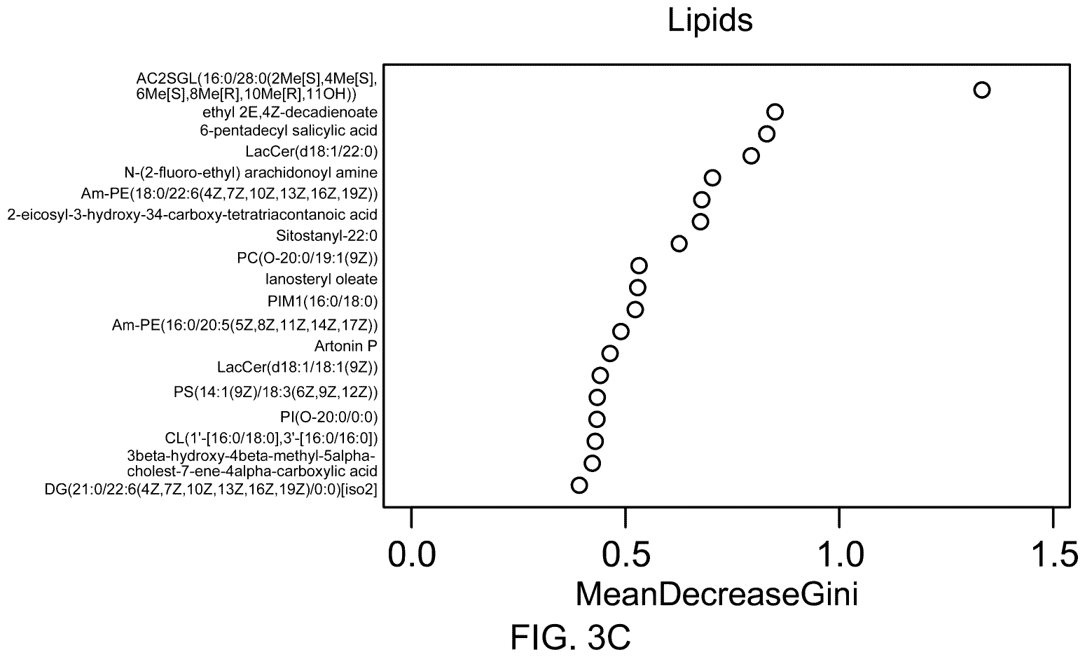

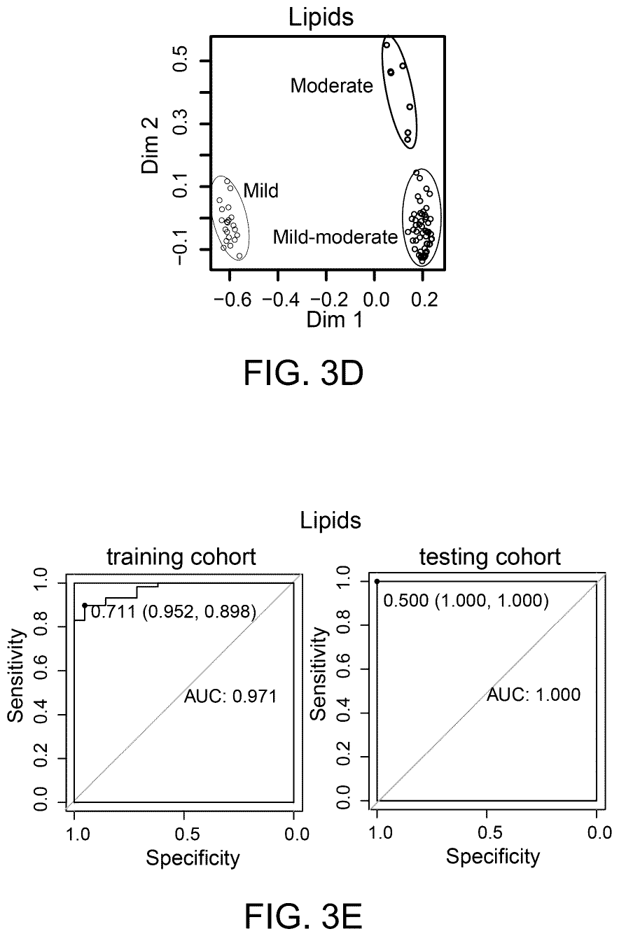

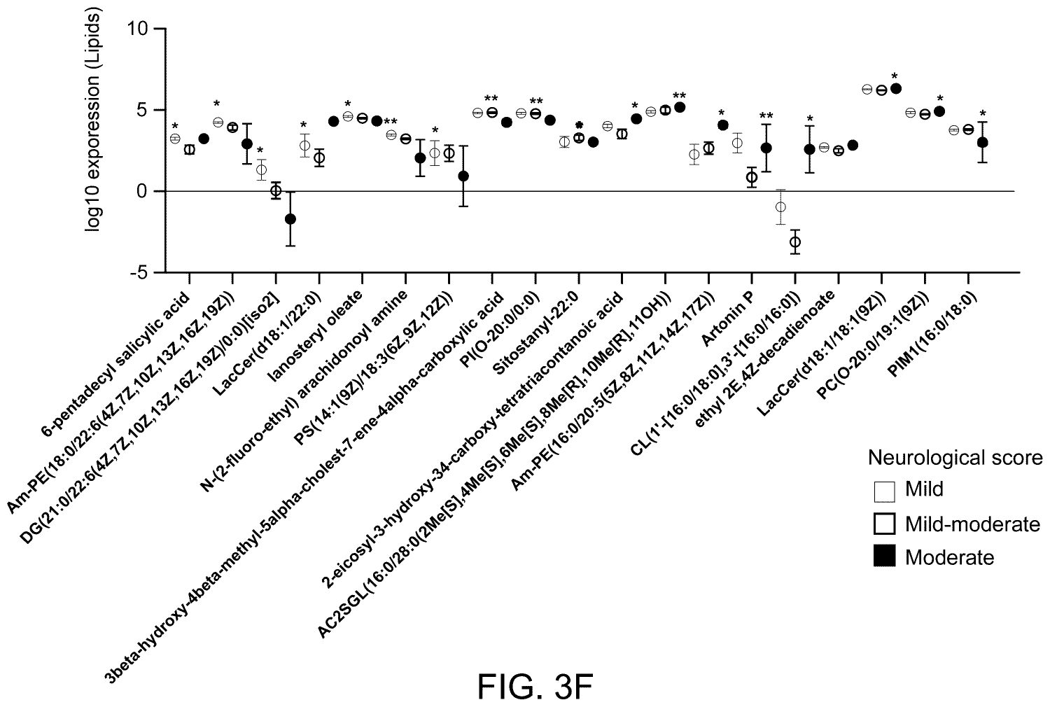

Results: As depicted in FIG. 3A, FIG. 3B, FIG. 3C, FIG. 3D, FIG. E and FIG. 3F, a set of core lipids was identified as potential biomarkers related to the severity of IS. These lipids include 2-eicosyl-3-hydroxy-34-carboxy-tetratriacontanoic acid, 3beta-hydroxy-4beta-methyl-5alpha-cholest-7-ene-4alpha-carboxylic acid, 6-pentadecyl salicylic acid, AC2SGL(16:0/28:0(2Me[S],4Me[S],6Me[S],8Me[R],10Me[R],11OH)), Am-PE(16:0/20:5(5Z,8Z,11Z,14Z,17Z)), Am-PE(18:0/22:6(4Z,7Z,10Z,13Z,16Z,19Z)), Artonin P, CL(1′-[16:0/18:0],3′-[16:0/16:0]), DG(21:0/22:6(4Z,7Z,10Z,13Z,16Z,19Z)/0:0)[iso2], ethyl 2E,4Z-decadienoate, LacCer(d18:1/18:1(9Z)), LacCer(d18:1/22:0), lanosteryloleate, N-(2-fluoro-ethyl) arachidonoyl amine, PC(O-20:0/19:1(9Z)), PI(O-20:0/0:0), PIM1(16:0/18:0), PS(14:1(9Z)/18:3(6Z,9Z,12Z)), Sitostanyl-22:0.

What is claimed is:

1. Biomarkers for severity of ischemic stroke, comprising protein markers, the protein markers comprise ACOX3 (Acyl-CoA oxidase 3), CAP1 (Cyclase-associated actin cytoskeleton regulatory protein 1), GRIPAP1 (GRIP1 associated protein 1), MAN1A1 (Mannosidase alpha class 1A member 1), UQCRC1 (Ubiquinol-cytochrome c reductase core protein 1), SULT1A2 (Sulfotransferase family 1A member 2), VNN1 (Vanin 1), BPGM (Bisphosphoglycerate mutase), COPS8 (COP9 signalosome subunit 8), HLA-DRB3 (Major histocompatibility complex, class II, DR beta 3), SPATS2L (Spermatogenesis associated serine-rich 2 like), VPS26A (VPS26 retromer complex component A), and DOCK1 (Dedicator of cytokinesis 1).

2. The biomarkers for severity of ischemic strokeaccording to claim 1, further comprising small molecule metabolite markers,and the small molecule metabolite markers comprise:
Valylleucine, (1S,2R,4As,6aS,6bR,8R,9R,10R,11R,12aR,12bR,14bS)-8,10,11-trihydroxy-9-(hydroxymethyl)-1,2,6a,6b,9,12a-hexamethyl-1,2,3,4,4a,5,6,6a,6b,7,8,8a,9,10,11,12,12a,12b,13,14b-icosahydropicene-4a-carboxylic acid, 1R-cis-3,3,5-Trimethylcyclohexyl ester 5-oxo-L-proline, Olopatadine n-oxide, Tetramethylene sulfoxide, Phenylacetaldehyde, Pentrinitrol, 13-OxoODE, 2-[[(2S)-1-[[(2S)-2-Carboxy-2-hydroxyethyl]amino]-1-oxo-3-phenylpropan-2-yl]amino]-4-phenylbutanoic acid, Beta-Citryl-L-glutamic acid, Desmethylmianserin, N,N-Didesmethyltramadol, Triethylene glycol dimethacrylate, and Uracil mustard.

3. The biomarkers for severity of ischemic strokeaccording to claim 1, further comprising lipid metabolite markers, and the lipid metabolite markers comprise: 2-eicosyl-3-hydroxy-34-carboxy-tetratriacontanoic acid, 3beta-hydroxy-4beta-methyl-5alpha-cholest-7-ene-4alpha-carboxylic acid, 6-pentadecyl salicylic acid, AC2SGL(16:0/28:0(2Me[S],4Me[S],6Me[S],8Me[R],10Me[R],11OH)), Am-PE(16:0/20:5(5Z,8Z,11Z,14Z,17Z)), Am-PE(18:0/22:6(4Z,7Z,10Z,13Z,16Z,19Z)), Artonin P, CL(1′-[16:0/18:0],3′-[16:0/16:0]), DG(21:0/22:6(4Z,7Z,10Z,13Z,16Z,19Z)/0:0)[iso2], ethyl 2E,4Z-decadienoate, LacCer(d18:1/18:1(9Z)), LacCer(d18:1/22:0), lanosteryloleate, N-(2-fluoro-ethyl) arachidonoyl amine, PC(O-20:0/19:1(9Z)), PI(O-20:0/0:0), PIM1(16:0/18:0), PS(14:1(9Z)/18:3(6Z,9Z,12Z)), and Sitostanyl-22:0.

4. A use of the biomarkers for severity of ischemic stroke according to claim 1 in the preparation of a diagnostic kit for assessing the severity of ischemic stroke.
